(19) United States
(12) Patent Application Publication
(10) Pub. No.: US 2016/0143515 A1
(43) Pub. Date: May 26, 2016

(54) OPTICAL SCANNING DEVICE AND LIGHT BEAM SCANNING METHOD

(71) Applicant: OLYMPUS CORPORATION, Tokyo (JP)

(72) Inventors: Atsuyoshi SHIMAMOTO, Tokyo (JP); Junichi NISHIMURA, Tokyo (JP)

(73) Assignee: OLYMPUS CORPORATION, Tokyo (JP)

(21) Appl. No.: 14/947,248

(22) Filed: Nov. 20, 2015

Related U.S. Application Data
(63) Continuation of application No. PCT/JP2014/002672, filed on May 21, 2014.

(30) Foreign Application Priority Data
May 21, 2013 (JP) 2013-107307

(51) Int. Cl. A61B 1/00; A61N 5/01; A61N 5/06

(52) U.S. Cl. CPC A61B 1/00172; A61N 5/0603; A61N 5/01

(57) ABSTRACT

An optical scanning endoscope apparatus, includes: an irradiation fiber having an emitting end thereof oscillatably supported and irradiating light from a light source part onto an object; and a drive mechanism for driving the emitting end so as to cause light from the light source to be irradiated onto the object, in which the apparatus has a first irradiation mode as an imaging mode (corresponding to t₁) for repeatedly scanning a desired region of the object with light from the light source and a second irradiation mode (corresponding to t₄) for irradiating, between the temporally-adjacent scans in the first irradiation mode, a designated region selected from the desired region of the object, and provides, when the second irradiation mode is started, the drive mechanism with an offset signal (I₀) for irradiating the designated region, and maintains the offset signal while repeating the irradiation in the second irradiation mode.

### CROSS REFERENCE TO RELATED APPLICATIONS

The present application is a Continuing Application based on International Application PCT/JP2014/002672 filed on May 21,2014, which, in turn, claims the priority from Japanese Patent Application No. 2013-107307 filed on May 21, 2013, the entire disclosures of which is incorporated herein by reference.

### TECHNICAL FIELD

The present disclosure relates to an optical scanning device and a light beam scanning method.

### BACKGROUND

There has been a demand for an optical scanning device such as a medical or industrial optical scanning endoscope apparatus or optical scanning microscope which has a plurality of functions in combination, not limited to image-observation of an object. For example, in the field of the medical optical scanning endoscope, there has been a desire for a single apparatus capable of carrying out both imaging and therapy such as laser ablation (endoscopic submucosal dissection (ESD)) without the need for reinserting the insertion portion of the endoscope. Further, in the industrial optical scanning endoscope, it may be desirable to carry out both imaging and laser measurement in a single apparatus. Further, the optical scanning microscope may desirably be capable of not only microscopically observing an object but also observing the object by giving light stimulus thereto.

In light thereof, the optical scanning device disclosed in Patent Literature 1 (PTL 1) uses a fiber having a tip end thereof oscillatably supported, so as to carry out both spiral scan of imaging laser and irradiation of therapeutic laser. FIG. 15 is a time chart for illustrating a beam scanning method when carrying out imaging and therapy parallel with each other. In the drawing, the frame i and the frame i+2 are imaging frames. In these frames, laser is scanned during the time (t₁) during which the laser irradiation position draws a spiral trajectory from the center with a gradually increasing radius, and the laser irradiation is stopped when the radius of the trajectory reaches maximum so that the amplitude returns to zero. At this time, a settling time (t₂) is needed to return the amplitude to zero. Such spiral scan allows for image observation of a circular region.

On the other hand, in the frames i+1 and i+3, therapeutic laser is irradiated onto a therapy region within the circular region. The therapy region is located out of the center of the circular region of spiral scan, and thus the optical scanning device applies an offset signal (direct current signal) to offset the laser irradiation position (t₃) when conducting therapeutic scan. When the laser irradiation position is offset, a periodic signal (alternating current signal) of small amplitude is applied with the offset signal being applied, so as to cause the laser irradiation position to make a small spiral scan around the offset position (t₄). This allows an arbitrary position within the circular region to be scanned with therapeutic layer. When the irradiation of therapeutic laser is completed, the offset signal is stopped and the laser irradiation position returns to the origin point (t₅).

15

### CITATION LIST

### Patent Literature

PTL 1: JP 2009-516568 A

### SUMMARY

An optical scanning device disclosed herein includes:

an irradiation fiber having an emitting end thereof oscillatably supported and irradiating an object with light from a light source; and

a drive mechanism for driving the emitting end of the irradiation fiber so as to cause light from the light source to be irradiated onto the object,

in which the optical scanning device has a first irradiation mode as an imaging mode and a second irradiation mode, the first irradiation mode being for repeatedly scanning a desired region of the object with light from the light source, the second irradiation mode being for irradiating, between the temporally-adjacent scans in the first irradiation mode, a designated region selected from the desired region of the object, and is configured to provide, when the second irradiation mode is started, the drive mechanism with an offset signal for irradiating the designated region, and, during when the irradiation of the second irradiation mode is being repeated, maintain the offset signal.

Preferably, the optical scanning device may further include a detection part for detecting light obtained from the object through irradiation of light from the light source,

in which the designated region may be selected based on an output from the detection part in the first irradiation mode.

In the first irradiation mode, the drive mechanism may preferably spirally scan the object.

The light source further includes an imaging light source and an application-specific light source, in which the first irradiation mode may use the imaging light source only, and the second irradiation mode may use at least the application-specific light source. Here, the object may be a biological tissue, and the application-specific light source may be a therapeutic light source. Alternatively, the application-specific light source may be a measurement light source such as, for example, near-infrared light for laser measurement, and the object may use, other than the biological tissue, various measuring objects.

The irradiation fiber may be a multicore fiber having a plurality of cores each for guiding light form the imaging light source and light from the application-specific light source, respectively.

The drive mechanism may be configured by including: a magnet attached to the irradiation fiber; and a plurality of electromagnetic coils arranged around the magnet. Alternatively, the drive mechanism may be configured by including a device for piezoelectric drive for driving the irradiation fiber.

The optical scanning device may further include: a display part for displaying, in the first irradiation mode, the object as an image based on an output from the detection part, and an input part for appointing the designated region on the image displayed on the display part, and may be configured to calculate the offset signal, based on the designated region appointed by the input part.

A light beam scanning method disclosed herein, includes:

vibratory driving an irradiation fiber having an emitting end thereof oscillatably supported, and repeatedly scanning a desired region of an object with light from a first light source;

detecting light obtained from the object scanned with light from the first light source, and generating image information; selecting, based on the image information, a designated region from the desired region;

displacing a scanning center of the irradiation fiber to the designated region; and

irradiating the designated region with light from a second light source, between the repetitive scans of the desired region with light from the first light source,

in which the displacement of the scanning center is maintained while repeating the irradiation of the designated region with light from the second light source.

Preferably, the scan may be a spiral scan.

The first light source may be an imaging light source, and the second light source may include an application-specific light source. Further, the object may be a biological tissue, and the application-specific light source may be a therapeutic light source. Alternatively, the application-specific light source may be a measurement light source.

The irradiation fiber may be a multicore fiber having a plurality of cores each for guiding light from the first light source and light from the second light source, respectively.

The displacement of the scanning center of the irradiation fiber may be made through driving the irradiation fiber with an electromagnetic force. Alternatively, the displacement of the scanning center of the irradiation fiber may be made through driving the irradiation fiber using a device for piezoelectric drive.

### BRIEF DESCRIPTION OF THE DRAWINGS

In the accompanying drawings:

FIG. 1 is a block diagram illustrating a schematic configuration of an optical scanning endoscope apparatus as an example of an optical scanning device according to Embodiment 1 disclosed herein;

FIG. 2 is an external view schematically illustrating the optical scanning endoscope main body of FIG. 1;

FIG. 3 is a sectional view illustrating a tip end part of the optical scanning endoscope main body of FIG. 2;

FIG. 4 is an enlarged perspective view of the drive mechanism of FIG. 3;

FIG. 5 illustrates a schematic configuration of the light source part of the optical scanning endoscope apparatus of FIG. 1;

FIG. 6 illustrates a schematic configuration of the detection part of the optical scanning endoscope apparatus of FIG. 1;

FIGS. 7A and 7B are graphs for illustrating scanning waveforms for carrying out imaging, where FIG. 7A shows a waveform in the x-axis direction, and FIG. 7B shows a waveform in the y-axis direction;

FIG. 8 illustrates a scanning trajectory for carrying out imaging;

FIG. 9 is a time chart showing a drive current to be applied to a drive mechanism when carrying out imaging and therapy parallel with each other;

FIGS. 10A and 10B are graphs for illustrating scanning waveforms in the frame i+2 of FIG. 9, where FIG. 10A shows a waveform in the x-axis direction, and FIG. 10B shows a waveform in the y-axis direction;

FIG. 11 shows a scanning trajectory in the xy plane in the frame i+2 of

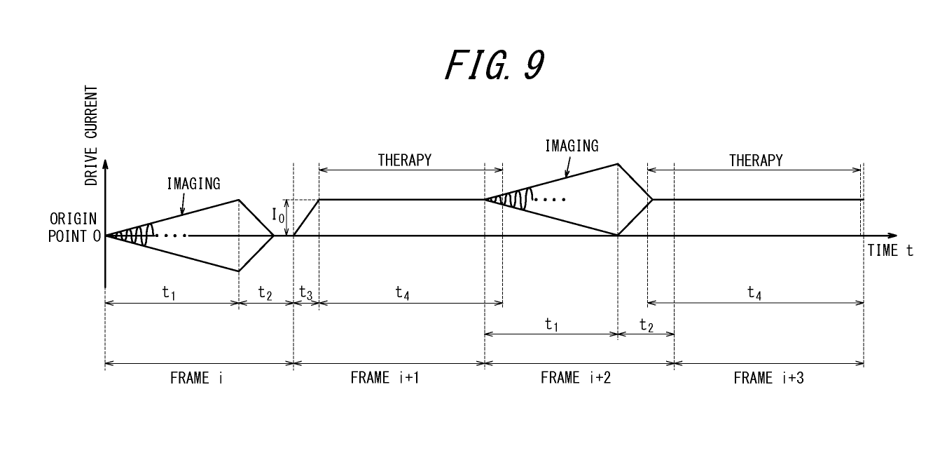

FIG. 9;

FIG. 12 is a sectional view of an irradiation multicore fiber of an optical scanning endoscope apparatus according to Embodiment 2 disclosed herein;

FIG. 13 is a view for illustrating offset caused in the scanning position when the irradiation multicore fiber of FIG. 12 is used;

FIG. 14 is a view for illustrating the scanning position of the therapeutic laser that has been offset into a designated region using the irradiation multicore fiber of FIG. 12; and

FIG. 15 is a time chart for illustrating beam scanning according to the conventional art when carrying out imaging and therapy parallel with each other.

### DETAILED DESCRIPTION

Hereinafter, Embodiments of the present disclosure will be illustrated with reference to the accompanying drawings.

### Embodiment 1

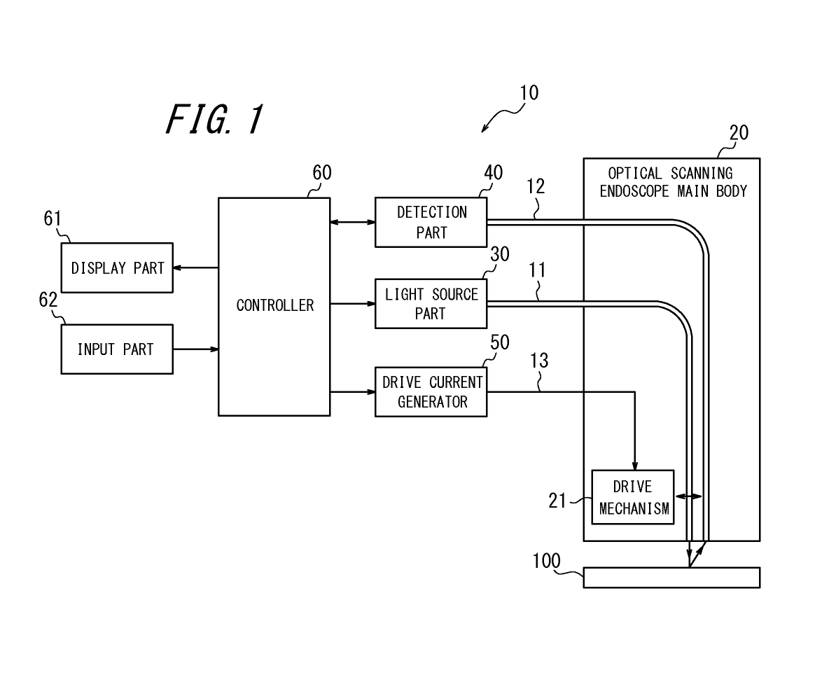

FIG. 1 is a block diagram illustrating a schematic configuration of an optical scanning endoscope apparatus 10 as an example of an optical scanning device according to Embodiment 1 disclosed herein. The optical scanning endoscope apparatus 10 is configured by including: an optical scanning endoscope main body 20; a light source part 30; a detection part 40; a drive current generator 50; a controller 60; a display part 61; and an input part 62. An irradiation fiber 11, which is a single mode fiber, optically connects between the light source part 30 and the optical scanning endoscope main body 20, and a plurality of detection fibers 12 formed of a multi-mode fiber connect between the detection part 40 and the optical scanning endoscope main body 20. A wiring cable is used to connect between the drive current generator 50 and the optical scanning endoscope main body 20, and between the controller 60 and each of the light source part 30, the detection part 40, the drive current generator 50, the display part 61, and the input part 62.

The irradiation fiber 11, the detection fibers 12, and the wiring cable 13 connecting between the drive current generator 50 and the optical scanning endoscope main body 20 are guided inside the optical scanning endoscope main body 20 to a tip end part. The irradiation fiber 11 is held within the tip end part of the optical canning endoscope main body 20 in such a manner that an emitting end of irradiation light (tip end part of the irradiation fiber 11, the part emitting light from the light source part 30) is capable of oscillating. The irradiation fiber 11 can irradiate an observation object 100 (object) with laser light from the light source part 30. Further, the wiring cable 13 is connected to a drive mechanism 21 in the tip end part of the optical scanning endoscope main body 20. The drive mechanism 21 can vibratory drive the tip of the irradiation fiber 11. The detection fibers 12 have an incident end part thereof arranged so as to cause light from the observation object 100 to be incident on a surface of the tip end part of the optical scanning endoscope main body 20, and guide the light received from the observation object 100 to the detection part 40.

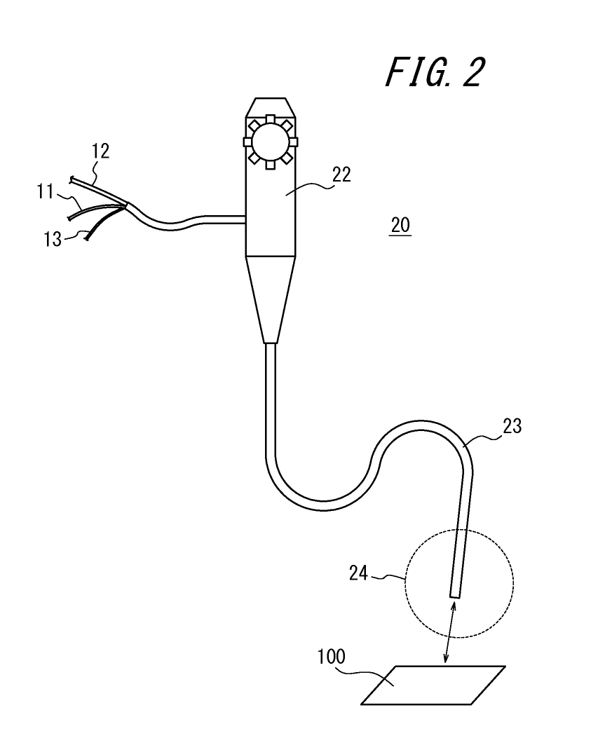

FIG. 2 is an overview schematically illustrating the optical scanning endoscope main body 20. The optical scanning endoscope main body 20 includes an operation portion 22 and an insertion portion 23 extending from one end of the operation portion 22. The irradiation fiber 11 from the light source part 30, the detection fibers 12 from the detection part 40, and the wiring cable 13 from the drive current generator 50 are each connected to the operation portion 22. The irradiation fiber 11, the detection fibers 12, the wiring cable 13 pass through the operation portion 22 to be guided, via the inside of the insertion part 23, to a tip end part 24 (circled by the broken line of FIG. 2) positioned at the tip of the insertion part 23.

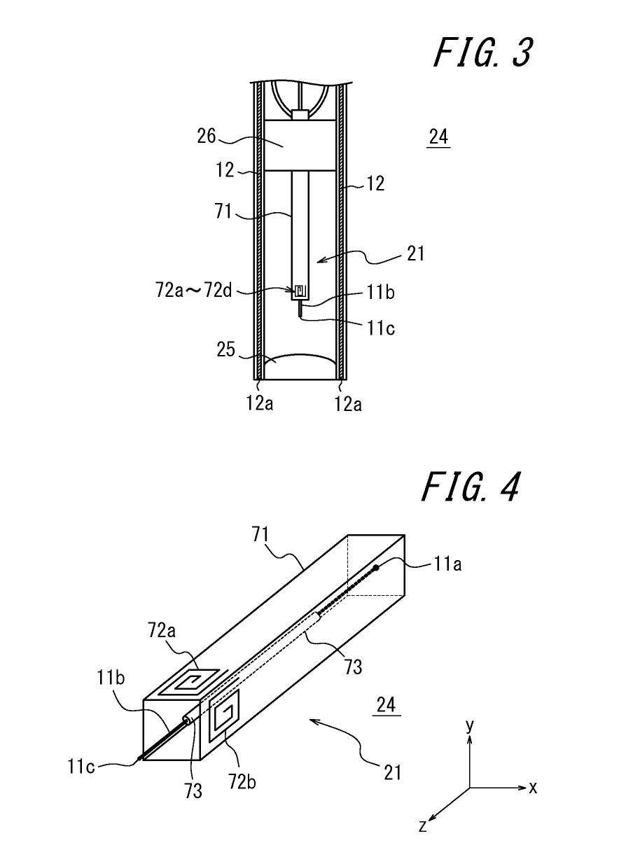

FIG. 3 is a sectional view illustrating the tip end part 24 of the optical scanning endoscope main body 20 of FIG. 2. The irradiation fiber 11 having passed through the insertion portion 23 is fixed and held, together with an angular tube 71 disposed to surround the irradiation fiber 11, to an attachment ring 26 attached to an inner wall of the tip end part 24 of the optical scanning endoscope main body 20. The irradiation fiber 11 is held in a cantilevered state at a fixed part 11a by means of the attachment ring 26 (see FIG. 4), and a part of the irradiation fiber 11 starting from the fixed part 11a to the emitting end 11c for emitting irradiation light is defined as an oscillating part 11b capable of oscillating within the angular tube 71. A projection lens 25 is disposed in front of the emitting end 11c of the irradiation fiber 11, so as to condense light emitted from the irradiation fiber 11 onto the observation object 100. Further, the plurality of detection fibers 12 are arranged so as to pass through the outer circumference of the insertion part 23 of the optical scanning endoscope main body 20, and have incident end parts 126a thereof arranged around the projection lens 25 disposed at the tip of the tip end part 24. Meanwhile, the angular tube 71 is formed of side faces of a rectangular prism, and has sheet-like electromagnetic coils 72a to 72d respectively disposed on each side face. The electromagnetic coils 72a to 72d constitute part of the drive mechanism 21.

FIG. 4 is an enlarged perspective view of the drive mechanism 21 of FIG. 3. The oscillating part 11b of the irradiation fiber 11 is attached with a permanent magnet 73 magnetized in the longitudinal direction of the irradiation fiber 11. The permanent magnet 73 is provided with a through hole for allowing the irradiation fiber 11 to penetrate therethrough along the axis of the columnar magnet. Further, the aforementioned electromagnetic coils 72a to 72d are disposed as being opposed to one of the poles of the permanent magnet 73. FIG. 4 merely illustrates the electromagnetic coils 72a and 72b only, but the magnetic coils 72c, 72d are also disposed on the other faces of the angular tube 71 opposing to the faces on which the magnetic coils 72a, 72b are disposed.

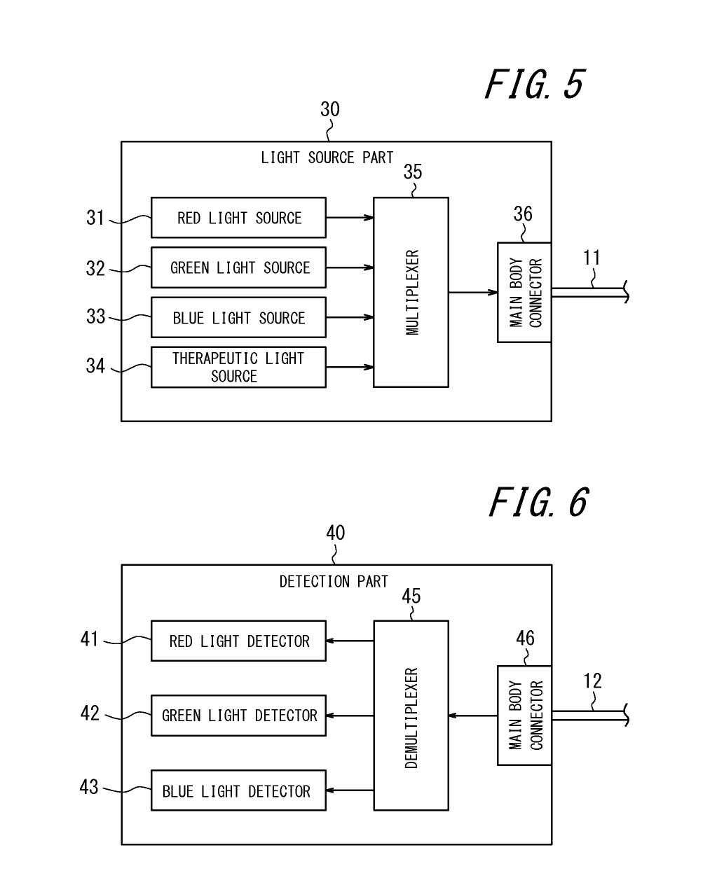

FIG. 5 illustrates a schematic configuration of the light source part 30 of the optical scanning endoscope apparatus 10 of FIG. 1. The light source part 30 includes: a red light source 31, a green light source 32, and a blue light source 33 for respectively emitting continuous wave (CW) laser light of three primary colors of red, green, and yellow; a therapeutic light source 34 (application-specific light source) for laser therapy; a multiplexer 35 for multiplexing lights from the respective light sources; and a main body connector 36 for guiding, to the irradiation fiber 11, light multiplexed in the multiplexer 35. Here, the red light source 31, the green light source 32, and the blue light source 33 constitute a first light source, while the therapeutic light source 34 constitutes a second light source. The red light source 31, the green light source 32, and the blue light source 33 may employ, for example, semiconductor lasers each having a wavelength of 640 nm, 532 nm, and 445 nm, respectively. The therapeutic light source 34 emits light of a different wavelength from those described above, and may be an ultraviolet (of up to 405 nm) laser. Further, the multiplexer 35 is configured by including, for example, a dichroic mirror and a fiber combiner. The main body connector 36 is configured by employing, for example, a fiber connector (FC) or a fiber coupling lens. Here, the red light source 31, the green light source 32, the blue light source 33, and the therapeutic light source 34 are connected to the controller 60 via wiring cables.

FIG. 6 illustrates a schematic configuration of the detection part 40 of the optical scanning endoscope apparatus 10 of FIG. 1. The detection part 40 includes: a red light detector 41, a green light detector 42, and a blue light detector43 each for each detecting light with wavelengths of red, green, and blue, respectively; a demultiplexer 45 for demultiplxing the detected light into light of each color; and a main body connector 46 for guiding the detected light from the detection fibers 12 into the detection part 40. The red light detector 41, the green light detector 42, and the blue light detector 43 each may employ, for example, a photodiode provided with a filter corresponding to the wavelength of each color. The demultiplexer 45 may be configured by using, for example, a dichroic mirror and a diffraction grating. Further, the main body connector 46 may be configured by using fiber connector (FC connector) and a fiber coupling lens. Here, the outputs of the red light detector 41, the green light detector 42, and the blue light detector 43 are connected to the controller 60 via wiring cables.

Further, the controller 60 of FIG. 1 synchronously controls the light source part 30, the detection part 40, and the drive current generator 50, while processing an electric signal output from the detection part 40 so as to synthesize an image and display the image on the display part 61. Further, various settings as to, for example, the scanning rate and the brightness of the displayed image can be made from the input part 62 to the optical scanning endoscope apparatus 10.

Next, description is given of imaging and therapy of a desired region of the observation object 100 as a biological tissue, using the optical scanning endoscope apparatus 10. First, in preparation for imaging the observation object 100, under the control of the controller 60, in the light source part 30, the multiplexer 35 multiplexes lights from the light source 31, the green light source 32, and the blue light source 33, and the multiplexed light is guided through the irradiation fiber 11 to the tip end part 24 of the optical scanning endoscope main body 20. At the same time, the drive current generator 50 applies, via the wiring cable 13, a current to each of the electromagnetic coils 72a to 72d constituting the drive mechanism 21. Here, the currents applied to the electromagnetic coils 72a, 72c and to the electromagnetic coils 72b, 72d are shifted in phase by 90 degrees and increase in amplitude over time. In this manner, the emitting end 11c of the irradiation fiber 11 vibrates so as to draw a spiral, and light emitted from the irradiation fiber 11 spirally scans a surface of the observation object 100 (first irradiation mode).

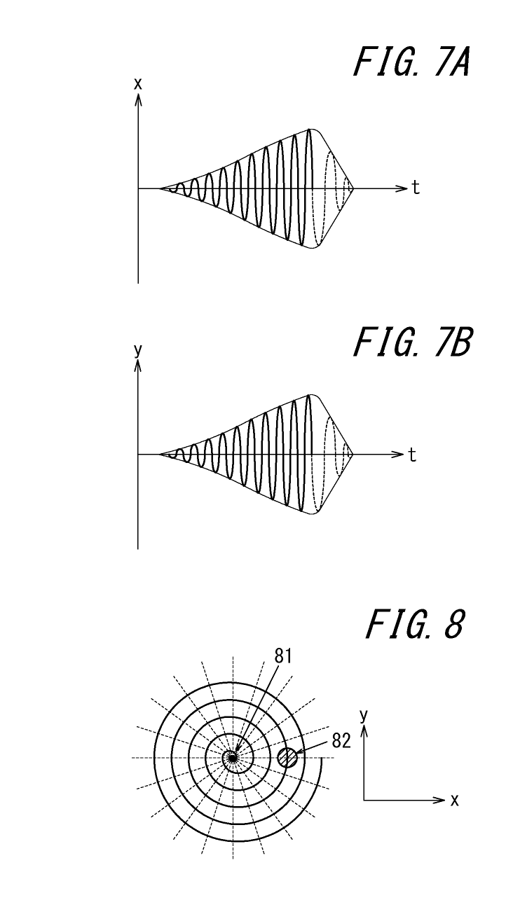

FIGS. 7A and 7B are graphs for illustrating scanning waveforms for carrying out imaging, where FIG. 7A shows a waveform in the x-axis direction, and FIG. 7B shows a waveform in the y-axis direction. Here, a direction in which the electromagnetic coils 72a and 72c face each other is defined as an y-axis direction, while a direction which the electromagnetic coils 72b and 72c face each other is defined as an x-axis direction. The waveform in the x-axis direction and the waveform in the y-axis direction are driven such that the waveforms are shifted in phase by approximately 90 degrees from each other. Through application of current to the electromagnetic coils 72a to 72d, the emitting end 11c of the irradiation fiber 11 vibrates with a gradually increasing amplitude, and thus, the scanning waveform on the observation object 100 also expands with time. Then, when the amplitude reaches a maximum value, the light source part 30 stops the emission of irradiation light, and the drive current from the drive current generator 50 rapidly returns to zero (which corresponds to the broken lines of FIGS. 7A and 7B). This way renders a scanning trajectory on the observation object 100 as shown in FIG. 8. In FIG. 8, the scanning center is denoted by 81.

The observation object 100, when irradiated with irradiation light, generates light (light to be detected) such as reflected light, scattered light, and fluorescence, and part of the light thus generated is incident on the incident end part 12a of the detection fibers 12 directed toward the observation object 100. The light to be detected is guided through the detection fibers 12 to the detection part 40, and in the detection part 40, demultiplexed by the demultiplexer 45 and detected, for each wavelength component, by the red light detector 41, the green light detector 42, and the blue light detector 43.

The controller 60 calculates information on the scanning position on the scanning path based on the waveform, intensity, and phase of a current to be applied by the drive current generator 50, and also obtains, based on an electric signal output from the detection part 40, pixel data of the observation object 100 at the relevant scanning position. The controller 60 sequentially stores information on the scanning position and the pixel data in a storage (not shown), carries out necessary processing such as interpolation processing after or during the scan to form an image of the observation object 100, and displays the image on the display part 61. The observation object 100 may be repeatedly scanned so as to obtain the image data as moving image data. In this manner, the affected area to be treated can be identified on the display part 61, and a designated region to be treated may be appointed from the region that has been spirally scanned. For example, a designated region 82 can be appointed, from the scanning range of FIG. 8 scanned by the irradiation light. Here, the designated region 82 may be appointed by a user of the optical scanning endoscope apparatus 10 by viewing the display part 61, or may be automatically specified through the analysis of the image by the controller 60.

Next, when the controller 60 instructs to treat the designated region 82, therapeutic laser is irradiated using the therapeutic light source 34 of the light source part 30, between temporally-adjacent repetitive scans for imaging, to thereby carry out imaging and therapy parallel with each other. Hereinafter, referring to FIG. 9, description is given of the drive current to be applied to the drive mechanism 21 in the case of carrying out imaging and therapy parallel with each other. Here, FIG. 9 is a time chart of the drive current for driving the electromagnetic coils 72b, 72d which apply a magnetic field in the x-axis direction, assuming a case where the designated region to be treated is found in the x-axis direction relative to the scanning center 81.

FIG. 9 shows a frame i, which is an imaging frame for carrying out imaging the state before therapy is started. One imaging frame lasts, for example, 1/30 of a second. When the therapeutic laser is not irradiated, this imaging frame is repeatedly carried out. Next, when instructed to irradiate the therapeutic laser, a therapy frame for carrying out therapy is executed, following the frame i, for the same duration (i.e., 1/30 of a second) as the imaging frame, and thereafter, imaging and therapy are alternately executed. That is, in FIG. 9, the frames i, i+2 are imaging frames and the frames i+1, i+3 are therapy frames.

The frame i requires settling time (t₂) for converging the amplitude to zero is needed, after imaging (t₁) through spiral scan. If the amplitude is abruptly returned from the maximum value to zero, the irradiation fiber 11 suffers undesirable vibration. Due to the time it takes to attenuate the vibration, a certain amount of time that is not negligible is needed for the settling time (t₂). Next, in the frame i+1, the electromagnetic coils 72b, 72d are applied with direct current (offset signal (I₀)) in order to displace the vibration center of the irradiation fiber 11 to a direction for irradiating the designated region 82 in the x-axis direction. After the time (t₃) it takes to complete the displacement (offset) of the vibration center, the therapeutic light source 34 of the light source part 30 irradiates therapeutic laser (t₄).

During the irradiation of therapeutic laser, the electromagnetic coils 72a to 72d of the drive mechanism 21 are applied with alternating current smaller in amplitude as compared with the case of imaging frame, so as to irradiate a partial region (designated region) within a desired region to be observed (second irradiation mode). Alternatively, the drive mechanism 21 may not be applied with alternating current during the therapy frame, and thus, a single spot in the designated region 82 may be irradiated (FIG. 9 shows the current to be applied to the drive mechanism 21 during therapy by a straight line for convenience).

After the therapy frame i+1, the imaging frame i+2 starts again, where the red light source 31, the green light source 32, the blue light source 33 of the light source part 30 emit irradiation light, so as to start spiral scan of the observation object 100. Here, the direct current being applied to the magnetic coils 72b, 72d is maintained without change, and therefore the position of the scanning center 81 on the observation object 100 is also maintained as displaced.

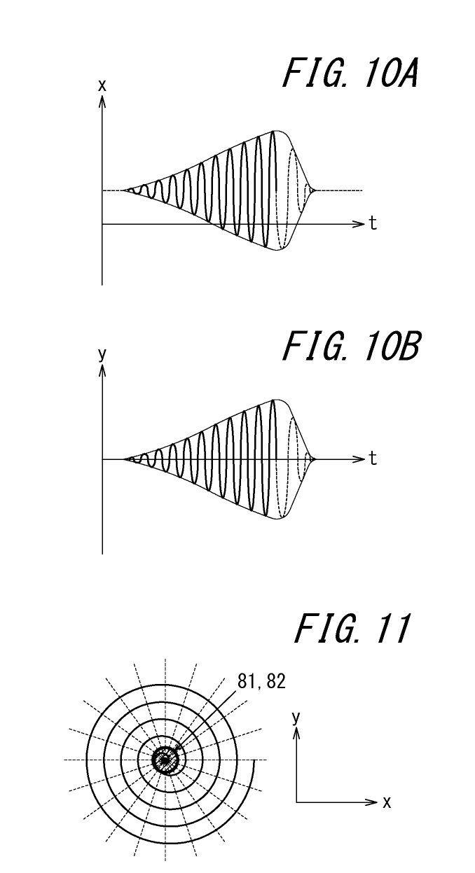

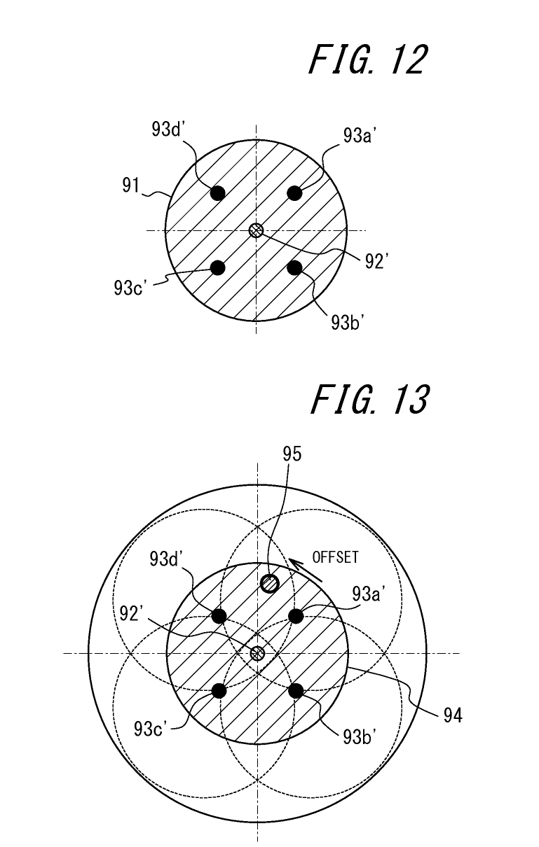

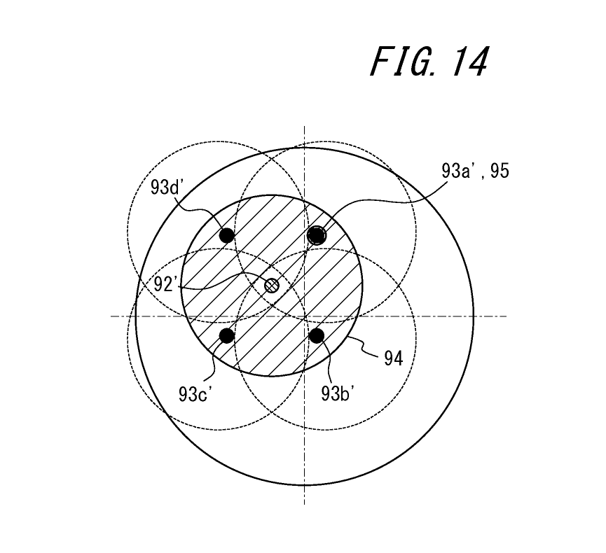

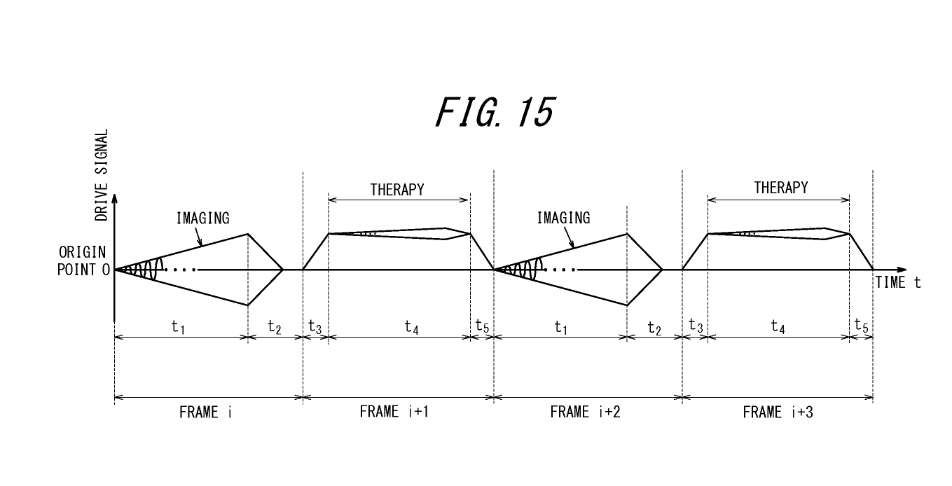

FIGS. 10A and 10B are graphs for illustrating scanning waveforms in the frame i+2 of FIG. 9, where FIG. 10A shows a waveform in the x-axis direction, and FIG. 10B shows a waveform in the y-axis direction. The vibration center has been displaced in the x-axis direction due to the direct-current component of an electromagnetic force acting between the electromagnetic coils 72b, 72d and the permanent magnet 73. For this reason, the scanning trajectory on the observation object 100 has the scanning center 81 that coincides with the designated region 82 as illustrated in FIG. 11, and thus the scanning trajectory is rendered as a spiral scan centering on the scanning center 81. Accordingly, in the image displayed on the display part 61, the designated region 82 to be treated is also displayed in the center.

Further, during when the amplitude of the spiral scan for imaging in the frame i+2 is smaller than the designated region 82 (that is, during when the scanning position stays inside the designated region 82 of FIG. 11), the irradiation of therapeutic laser can be continued simultaneously with imaging. In the spiral scan, the sampling density increases in the vicinity of the scanning center 81 where the amplitude is small, as compared with the periphery of the scan. In the case of conducting imaging only, some of the pixels in the vicinity of the scanning center go to waste without being used. However, in the irradiation of therapeutic laser, scanning at a position where the amplitude is smaller can effectively be utilized. Therefore, as shown in FIG. 9, the therapy time (t₄) with the user of therapeutic laser may partially overlap with the imaging time (t₁) after the offset in the frame i+1 to the initial stage of the spiral scan in the frame i+2. As a result, the irradiation time of therapeutic laser can be extended as compared with the conventional art.

Further, in transition to the therapy frame of the frame i+3 after the imaging frame i+2, there is no need to displace the vibration center, which eliminates the need to change the direct current (offset signal (I₀)) to be applied to the magnetic coils 72b, 72d of the drive mechanism 21. Therefore, the time (t₃) to drive offset in the frame i+1 is no longer needed. Based on our calculation from the equation of motion, it takes approximately 20 ms for the conversion of the irradiation fiber 11 after application of the offset signal. Therefore, for example, assuming that the frame length of one frame is about 33 ms (30 fps), the present disclosure is extremely effective to ensure the therapy time (t₄). Further, after the imaging time (t₁) through spiral scan, the irradiation of therapeutic layer can be started as early as when the vibration of the irradiation fiber 11 has been roughly converged in the settling time (t₂) for converging the amplitude to zero. This can ensure a further longer therapy time t₄ in the frame i +3, and thus the irradiation intensity of therapeutic laser can be increased as a whole.

Even in the rest of frames after the frame i+3, the direct current (offset signal (I₀) to be applied to the magnetic coils 72a, 72d of the drive mechanism 21 is maintained until the therapy is stopped based on the user instruction made via the input part 62 or the judgment made by the controller 60 based on the image data acquired through imaging.

As described above, according to Embodiment 1, the disclosed device has a first irradiation mode and a second irradiation mode, the first irradiation mode being for repeatedly scanning, for imaging, a desired region of the observation object 100 with irradiation light from the red light source 31, the green light source 32, and the blue light source 33 of the light source part 30, the second irradiation mode being for irradiating, based on the output signal, the designated region 82 selected from the desired region, with therapeutic laser from the therapeutic light source 34, in between the temporally-adjacent scans for imaging. In the device, when instructed to start the second irradiation mode, the offset signal (I₀) is applied as direct current to the electromagnetic coil of the drive mechanism 21 for irradiating the designated region 82, and the offset signal (I₀) is maintained during the repetition of the second irradiation mode, which eliminates the need for the time it takes to switch the scanning center (on/off of the offset) between the imaging scan and the therapeutic laser irradiation, and allows to increase the time for irradiating therapeutic laser light per unit frame, making it possible to efficiently irradiating therapeutic light onto the designated region 82 within the imaging region.

Further, when the offset signal (I₀) is thus maintained, the scanning center and the center of the designated region 82 to be treated coincide with each other, so that therapeutic laser can be simultaneously irradiated when the amplitude of the imaging spiral scan is small, with the result that the irradiation time of therapeutic laser can be extended. In this manner, total energy of irradiation of therapeutic laser per unit frame can be intensified while keeping high the frame rate of the imaging frame.

In Embodiment 1, the scanning center 81 is displaced to the direction of the designated region 82, which is in the x-axis direction in which a pair of the electromagnetic coils 72b, 72d facing each other, but the scanning center 81 may be displaced in any direction in the xy plane. The electromagnetic coils 72a, 72c in the y-axis direction and the electromagnetic coils 72b, 72d in the x-axis direction may be applied with direct current components corresponding to the displacement direction, to thereby displace the scanning center 81 in a desired direction. The drive mechanism 21 may not be limitedly configured as an angular tube, but may employ various configurations. For example, a cylindrical tube may be used in place of the angular tube, and electromagnetic coils may be arranged thereon. Further, the red light source 31, the green light source 32, and the blue light source 33 for imaging may be turned off outside the imaging time (t₁), or may always be turned on. Further, in order to detect light to be obtained from the observation object 100, the detection fibers 12 and the detection part 40 may be replaced by, for example, an optical detection element at the tip end part 24 of the optical scanning endoscope main body 20 and an output signal thereof may be transmitted to the controller through a wiring cable.

### Embodiment 2

FIG. 12 is a sectional view of an irradiation multicore fiber of an optical scanning endoscope apparatus according to Embodiment 2 disclosed herein. Embodiment 2 uses an irradiation multicore fiber 91 in place of the irradiation fiber 11 as a single core fiber of Embodiment 1. The irradiation multicore fiber 91 has an imaging core 92 and four therapeutic cores 93a to 93d. The imaging core 92 is positioned in the center of the irradiation multicore fiber 91, and the therapeutic cores 93a to 93d are arranged at substantially equal distance from the imaging core while being spaced apart from one another by 90 degrees.

The light source part in this case, unlike the light source part 30 of FIG. 5, is configured to multiplex, by mean of the multiplexer 35, the red light source 31, the green light source 32, and the blue light source 33, and connect the light sources to the imaging core 92, while providing four therapeutic light sources 34, which are each connected to the therapeutic cores 93a to 93d, respectively. The rest of the configuration is similar to that of Embodiment 1, and thus the same or corresponding constituent elements are denoted by the same reference symbols to omit the description thereof.

Next, description is given of the observation and therapy procedure to be performed when using the irradiation multicore fiber 91 of FIG. 12 to irradiate the observation object 100. First, in the imaging frame, the imaging core 92 is used to scan irradiation light on the observation object 100 as in the case shown in FIG. 9. FIG. 13 illustrates how the observation object 100 is scanned by the irradiation multicore fiber 91 of FIG. 12. This shows that the irradiation multicore fiber 91 is spirally scanned so as to scan an imaging region 94 centering on the scanning center of the imaging core 92.

(Reference numerals 92′, 93a′ to 93d′ each denote an irradiation position of light from the imaging core 92, and the therapeutic cores 93a to 93d, respectively, when stationary.) In this manner, pixel data on each scanning position on the imaging region 94 is detected by the detection part 40, which is used to generate an image by the controller 60, and the image is displayed on the display part 61. Then, a designated region 95 to be treated is appointed, based on a user selection made from the input part 62 or by judging from image data acquired by the controller 60.

Next, a description is given of a case of irradiating the designated region 95 with therapeutic laser, between temporally-adjacent imaging frames. In this case, selected is one of the therapeutic cores 93a to 93d that irradiates a position closest to the designated region 95 when the scan is stopped, rather than the imaging core 92 positioned in the center of the irradiation multicore fiber 91. In the example of FIG. 13, the position irradiated by the therapeutic core 93a is closest to the designated region 95, and thus, in the therapy frame after the settling time of spiral scan in the imaging frame, the drive mechanism 21 is applied with direct current (offset signal) so that the therapeutic core 93a scans the designated region 95. FIG. 14 is a view for illustrating the irradiation position 93a ′ of the therapeutic laser that has been displaced to the center of the designated region 95 using the irradiation multicore fiber 91 of FIG. 12. In this state where the offset signal is applied, an alternating current with a small amplitude may further be applied to the drive mechanism, to thereby irradiate the designated region 95 with therapeutic laser.

After the irradiation of therapeutic laser, the frame again returns to the imaging frame, where imaging irradiation light is irradiated from the imaging core 92 and spiral scan is performed, while maintaining the direct current signal in the drive mechanism 21. Thereafter, therapeutic laser is irradiated between temporally-adjacent imaging frames until the irradiation of therapeutic laser is stopped, and during the irradiation of the therapeutic laser, the offset signal applied to the drive mechanism 21 is maintained. In this case, unlike in Embodiment 1, the scanning center in imaging does not coincide with the center of designated regions 95.

According to Embodiment 2, as in the case of Embodiment 1, once the scanning position is displaced in the first therapeutic frame, there is no need for the time it takes to switch the scanning center position (on/off of the offset) between the imaging frame and the therapy frame, which can extend the time for irradiating therapeutic layer, making it possible to efficiently irradiating therapeutic light onto the designated region 82 in the imaging region. Further, of the four therapeutic cores 93a to 93d, there may be selected one that irradiates a position closest to the designated region 82, and thus, the distance of the displacement of the scanning center caused by direct current (offset signal) applied to the drive mechanism 21 may be reduced to small. Therefore, as compared with the case of using a single core fiber, the drive current may be reduced, allowing for more stable scan.

In Embodiment 2, the number and arrangement of the imaging core 92 and the therapeutic cores 93a to 93d in the irradiation multicore fiber 91 are merely examples, and the cores may be arranged in various other ways. Further, the imaging core 92 and the therapeutic cores 93a to 93d may need not to be fixed, and the imaging light source and the therapeutic light source may be switched in the light source part 30. Further, there may be provided only one therapeutic light source 34, which may be switchably connected to one of the therapeutic cores 93a to 93d to be used.

It should be noted that the present disclosure is not limited only to Embodiments described above, and may be subjected to various modifications and alterations. For example, the drive mechanism is not limited to the one using an electromagnetic coil and a magnet, and may be the one using a piezoelectric element (device for piezoelectric drive). For example, four piezoelectric elements extendible and contractible in a direction along the axis of the irradiation fiber may be disposed as being opposed to each other either in the x direction and the y direction of the oscillating part of the irradiation fiber, and opposing piezoelectric elements may be applied with vibration voltage of reversed phase, to thereby vibratory drive the irradiation fiber. In this case, in place of the drive current generator, a drive voltage generator may be provided for supplying drive voltage to the piezoelectric elements under the control of the controller.

Further, the scanning method for imaging is not limited to spiral scan, and may be applied to raster scan and Lissajous scan. In this case as well, there is no need to switch the scanning position every between the first irradiation mode for imaging and the second irradiation mode, which allows for efficient irradiation in the second irradiation mode. However, in the spiral scan, the irradiation direction of irradiation light always returns to the origin point at the end of scan, and thus, the present disclosure may become more effective when applied to spiral scan because the designated region can be immediately observed once the scanning center is shifted to the designated region.

Further, as the first irradiation mode, in addition to or in place of the light from the imaging light sources of blue, green, and yellow, light of a suitable wavelength (measurement light) for narrow band imaging (NBI) observation, fluorescent observation may be irradiated, so as to subject an image of an object, such as an affected area of a human body, to visual observation or image analysis, to thereby identify a site to be treated and determine a designated region for conducting therapy. As the second irradiation mode, in addition to or in place of the therapeutic light, light of measurement wavelength described above may be irradiated, so as to measure the designated region. As described above, the present disclosure is applicable to the measurement application, not limited to the therapeutic application, and the measurement may be conducted alone or together with the therapy.

Further, the disclosed device and method may be applied not only to a therapeutic optical scanning endoscope apparatus but to various devices, such as an industrial optical scanning endoscope for carrying out imaging and measurement of a specific region, and an optical scanning microscope for carrying out imaging and providing light stimulus to a specific region.

1. An optical scanning device, comprising:
an irradiation fiber having an emitting end thereof oscillatably supported and irradiating an object with light from a light source; and
a drive mechanism for driving the emitting end of the irradiation fiber so as to cause light from the light source to be irradiated onto the object,
wherein the optical scanning device has a first irradiation mode as an imaging mode and a second irradiation mode, the first irradiation mode being for repeatedly scanning a desired region of the object with light from the light source, the second irradiation mode being for irradiating, between the temporally-adjacent scans in the first irradiation mode, a designated region selected from the desired region of the object, and
wherein the optical scanning device is configured to provide, when the second irradiation mode is started, the drive mechanism with an offset signal for irradiating the designated region, and, during when the irradiation of the second irradiation mode is being repeated, maintain the offset signal.

2. The optical scanning device according to claim 1, further comprising a detection part for detecting light obtained from the object through irradiation of light from the light source,
wherein the designated region is selected based on an output from the detection part in the first irradiation mode.

3. The optical scanning device according to claim 1, wherein, in the first irradiation mode, the drive mechanism spirally scans the object.

4. The optical scanning device according to claim1,
wherein the light source comprises an imaging light source and an application-specific light source,
wherein the first irradiation mode uses the imaging light source only, and the second irradiation mode uses at least the application-specific light source.

5. The optical scanning device according to claim 4, wherein the object is a biological tissue, and the application-specific light source is a therapeutic light source.

6. The optical scanning device according to claim 4, wherein the application-specific light source is a measurement light source.

7. The optical scanning device according to claim 4, wherein the irradiation fiber is a multicore fiber having a plurality of cores each for guiding light form the imaging light source and light from the application-specific light source, respectively.

8. The optical scanning device according to claim 1, wherein the drive mechanism is configured by including: a magnet attached to the irradiation fiber; and a plurality of electromagnetic coils arranged around the magnet.

9. The optical scanning device according to claim 1, wherein the drive mechanism is configured by including a device for piezoelectric drive for driving the irradiation fiber.

10. The optical scanning device according to claim 1, further comprising a display part for displaying, in the first irradiation mode, the object as an image based on an output from the detection part, and an input part for appointing the designated region on the image displayed on the display part,
the optical scanning device being configured to calculate the offset signal, based on the designated region appointed by the input part.

11. A light beam scanning method, comprising:
vibratory driving an irradiation fiber having an emitting end thereof oscillatably supported, and repeatedly scanning a desired region of an object with light from a first light source;
detecting light obtained from the object scanned with light from the first light source, and generating image information;
selecting, based on the image information, a designated region from the desired region;
displacing a scanning center of the irradiation fiber to the designated region; and
irradiating the designated region with light from a second light source, between the repetitive scans of the desired region with light from the first light source,
wherein the displacement of the scanning center is maintained while repeating the irradiation of the designated region with light from the second light source.

12. The light beam scanning method according to claim 11, wherein the scan is a spiral scan.

13. The light beam scanning method according to claim 11, wherein the first light source is an imaging light source, and the second light source includes an application-specific light source.

14. The light beam scanning method according to claim 13, wherein the object is a biological tissue, and the application-specific light source is a therapeutic light source.

15. The light beam scanning method according to claim 13, wherein the application-specific light source is a measurement light source.

16. The light beam scanning method according to claim 11, wherein the irradiation fiber is a multicore fiber having a plurality of cores each for guiding light from the first light source and light from the second light source, respectively.

17. The light beam scanning method according to claim 11, wherein the displacement of the scanning center of the irradiation fiber is made through driving the irradiation fiber with an electromagnetic force.

18. The light beam scanning method according to claim 11, wherein the displacement of the scanning center of the irradiation fiber is made through driving the irradiation fiber using a device for piezoelectric drive.
